(19) United States
(12) Patent Application Publication
(10) Pub. No.: US 2006/0251690 A1
(43) Pub. Date: Nov. 9, 2006

(54) Skin enrichment using CoQ10 as the delivery system

(75) Inventors: Bruce H. Lipshutz, Goleta, CA (US); Deanne Dolnick, North Hollywood, CA (US)

Correspondence Address: MORGAN, LEWIS & BOCKIUS LLP (SF), 2 PALO ALTO SQUARE, 3000 El Camino Real, Suite 700, PALO ALTO, CA, 94306, US

(73) Assignee: Zymes, LLC, Hasbrouck Heights, NJ (US)

(21) Appl. No.: 11/397,380

(22) Filed: Apr. 3, 2006

Related U.S. Application Data
(60) Provisional application No. 60/667,380, filed on Apr. 1, 2005.
(60) Provisional application No. 60/668,273, filed on Apr. 5, 2005.

(51) Int. Cl. A61K 31/7048; A61K 31/65; A61K 31/203; A61K 31/192; A61K 31/19; A61K 31/07; A61K 31/12

(52) U.S. Cl. 424/401; 514/690; 514/559; 514/557; 514/154; 514/568; 514/29; 514/574; 514/725

(57) ABSTRACT

The present invention provides several skin enrichment formulations comprising ubiquinones. The present invention provides a composition for the treatment of acne, topical antimicrobial ointment, sunscreening agents, facial cleansing, reduction of skin inflammation, firming skin tone, cosmetic foundation, skin protection, lip skin protection and skin cream. In a preferred embodiment, the composition contains Coenzyme Q₁₀.

### CROSS-REFERENCE TO RELATED APPLICATIONS

This application claims priority to U.S. Provisional Application No. 60/668,273, filed Apr. 5, 2005 and to U.S. Provisional Application No. 60/667,380, filed Apr. 1, 2005, the disclosures of which are incorporated herein by reference for all purposes.

### BACKGROUND OF THE INVENTION

The ubiquinones, also commonly called coenzyme Qₙ (n=1-12), constitute essential cellular components of many life forms. In humans, CoQ₁₀ is the predominant member of this class of polyprenoidal natural products and is well-known to function primarily as a redox carrier in the respiratory chain and it is the form most studied for therapeutic potential (Lenaz, COENZYME Q. BIOCHEMISTRY, BIOENERGETICS, AND CLINICAL APPLICATIONS OF UBIQUINONE, Wiley-Interscience: New York (1985); Trumpower, FUNCTION OF UBIQUINONES IN ENERGY CONSERVING SYSTEMS, Academic Press, New York (1982); Thomson, R. H., NATURALLY OCCURRING QUINONES, 3rd ed., Academic Press, New York (1987); Bliznakov et al., THE MIRACLE NUTRIENT COENZYME Q₁₀, Bantom Books, New York (1987)).

Coenzyme Q plays an essential role in the orchestration of electron-transfer processes necessary for respiration. Almost all vertebrates rely on one or more forms of this series of compounds that are found in the mitochondria of every cell (i.e., they are ubiquitous, hence the alternative name “ubiquinones”). Although usually occurring with up to 12 prenoidal units attached to ap-quinone headgroup, CoQ₁₀ is the compound used by humans as a redox carrier.

An underappreciated fact of CoQ₁₀ is when less than normal levels of CoQ₁₀ are present, the body must construct its CoQ₁₀ from lower forms obtained through the diet. Unfortunately, at some point in everyone's life span, the efficiency of the CoQ₁₀ synthesis machinery begins to drop (Blizakov et al., supra). The consequences of this in vivo deterioration can be substantial since levels of CoQ₁₀ have been correlated with sensitivity to infection, strength of heart muscle, and metabolic rates tied to energy levels and vigor. Thus, as people age, supplementation with CoQ₁₀ is increasingly necessary for the maintenance of youthfulness and health.

In the United States, CoQ₁₀ is considered a dietary supplement, sold typically in health food stores or through mail order houses at more or less reasonable prices. CoQ₁₀ is typically produced through well-established fermentation and extraction processes (e.g., Sasikala et al., Adv. Appl. Microbiol., 41:173 (1995); U.S. Pat. Nos. 4,447,362; 3,313,831; and 3,313,826). However, new cost effective methods of synthesis are now available. Such methods are disclosed in U.S. Pat. No. 6,852,895; U.S. Pat. No. 6,545,184; co-pending U.S. patent application Ser. No. 10/973,158 filed Oct. 24, 2004, and co-pending U.S. patent application Ser. No. 11/003,544, filed Dec. 3, 2004 each of which is incorporated herein by reference in its entirety.

As the population ages, and concerns with the maintenance of youthful vigor and appearance continue to grow, supplementation of the natural levels of CoQ₁₀ will become increasingly important. Furthermore, the demand for cosmetic and medical formulations that preserve skin health and youthful appearance will undoubtedly increase. Thus, there is a need in the art for products that provide CoQ₁₀ to supplement the natural levels in the body. Furthermore, there is a need in the art for delivery systems that provide enhanced absorption of secondary cosmetic and medical products which function to maintain skin health and youthful vigor. Fortunately, the invention provides for these and other needs.

### SUMMARY OF THE INVENTION

The present invention provides in a first aspect, a dermatological formulation comprising a compound according to Formula (I): 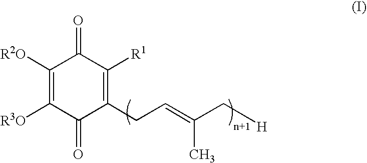
wherein R¹, R² and R³ are members independently selected from substituted and unsubstituted C₁-C₆ alkyl groups and n is an integer from 0 to 19. In one embodiment, n is 9. In another embodiment R¹, R² and R³ are methyl groups. In another embodiment, the compound is in a reduced form.

In some embodiments, the dermatological formulation further comprises a representative solubilizing agent according to Formula (II): 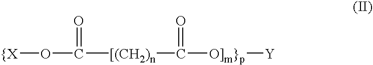
wherein X is a residue of a hydrophobic moiety, selected from sterols, tocopherols, and derivatives thereof; Y is a residue of a hydrophilic moiety selected from polyethers, polyalcohols, and derivatives thereof; p is 1 or 2; m is 0 or 1; and n is an integer greater than or equal to 0. When p and m are equal to 1 and the hydrophobic moiety is cholesterol, n is greater than 4 and not equal to 8. When p and m are equal to 1, and the hydrophobic moiety is (+)-α-tocopherol, n is not equal to 2. In one embodiment, when the hydrophobic moiety is a member selected from cholesterol, 7-dehydrocholesterol, campesterol, sitosterol, ergosterol, stigmasterol, α-tocopherol, β-tocopherol, γ-tocopherol, and δ-tocopherol, the hydrophilic moiety is a polyether or a cyclodextrin.

In one embodiment, the invention provides compositions for the treatment of acne and enhanced dermatological health. These formulations comprise an anti-acne therapeutic, and a compound according to Formula (I). In one embodiment, the anti-acne therapeutic is a member selected from a retinoid compound, azelaic acid, adapalene, salicylic acid, glycolic acid, benzoyl peroxide, and combinations thereof.

In one embodiment, the anti-acne therapeutic is a retinoid compound that is a member selected from retinol, a compound according to Formula (III) and a compound according to Formula (IV):

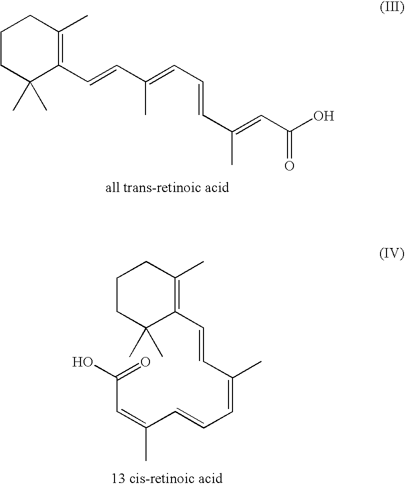

In one embodiment, the anti-acne composition provides between about 1% to about 1000% better treatment of acne over formulations lacking a compound according to Formula (I). In another embodiment, the anti-acne composition provides between about 5% to about 500% better treatment of acne. In still another embodiment, the anti-acne composition provides between about 25% to about 100% better treatment of acne.

In a related embodiment, the invention provides a composition for reducing skin inflammation and firming skin tone. The composition comprises a compound according to Formula (I). In one embodiment, the composition further comprises cortisone. In one embodiment, the skin inflammation treated by the composition is associated with an inflammatory dermatosis. In one embodiment, the inflammatory dermatosis is Acne Rosacea. In some embodiments, the composition further comprises a compound according to Formula (II).

In another embodiment, the invention provides a composition for enhanced performance of a topical antimicrobial ointment comprising a therapeutically effective topical antimicrobial agent, and a compound according to Formula (I). In one embodiment, the antimicrobial agent is selected from alcohol, neosporin, polysporin, benzalkonium chloride, chlorhexidine, hexachlorophine, fusidic acid, neomycin, oxytetracyclin, mupirocin, flucloxacillin, acyclovir and combinations thereof. In another embodiment, the antimicrobial ointment composition further comprises an analgesic. In another embodiment, antimicrobial ointment composition further comprises an anti-inflammatory agent. In another embodiment, the anti-inflammatory agent further comprises cortisone. In some embodiments, the antimicrobial ointment composition further comprises a compound according to Formula (II).

In another embodiment, the invention provides a sunscreen composition that also provides enhanced dermatological health. The sunscreen composition comprises an effective sunscreening agent, and a compound according to Formula (I). In some embodiments, the sunscreen formulation further comprises a compound according to Formula (II). In one embodiment, the sunscreen composition is comprised within a formulation for a cosmetic foundation, a lip balm, or a lipstick.

In another embodiment, the invention provides a composition for cleansing and improved dermatological health. The cleansing composition comprises an effective cleanser and a compound according to Formula (I). In some embodiments, the cleansing composition further comprises a compound according to Formula (II).

In one embodiment, the cleansing composition is comprised within a formulation for a product selected from a facial mask and a bar soap. In one embodiment, the cleansing composition is comprised within a formulation for a facial mask, and further comprises glacial silt or glacial clay as the excipient. In one embodiment, the glacial silt is comprised of silt particles whose average diameter is in a range from between about 0.004 mm. to about 0.063 mm.

In another embodiment, the invention provides a method for delivering a composition comprising a compound together with a compound according to Formula (I). The method comprises providing the ubiquinone compound as the transdermal delivery system. In one embodiment, the method comprises providing a compound according to Formula (I) together with a compound according to Formula (II), thereby forming an alternative transdermal delivery system. In one embodiment the transdermal delivery system is a patch that releases the compound together with the ubiquinone over time (e.g., a “CoQ₁₀ patch”).

In another embodiment the invention provides a cosmetic foundation that also improves dermatological health. The cosmetic foundation comprises a compound according to Formula (I). In some embodiments the cosmetic foundation further comprises a compound according to Formula (II).

In another embodiment the invention provides a skin lotion comprising a compound according to Formula (I) and hyaluronic acid. In one embodiment, the compound according to Formula (I) improves penetration of the hyaluronic acid into skin cells. In another embodiment, the hyaluronic acid penetrates skin cells between about 1% to about 1000% better than hyaluronic acid in a lotion formulation lacking a compound according to Formula (I).

In another embodiment, the invention provides a composition for skin protection that comprises a compound according to Formula (I) and a compound that is a member selected from mineral oil and petroleum jelly.

Other objects and advantages of the invention will be apparent to those of skill in the art from the detailed description that follows.

### DETAILED DESCRIPTION OF THE INVENTION

Definitions

The term “COQ₁₀” or Coenzyme Q₁₀, Q₁₀, vitamin Q₁₀, CoQ(50), ubiquinone, ubidecarenone or 2,3 dimethoxy-5 methyl-6 decaprenyl benzoquinone or the like, refers to a compound according to the formula 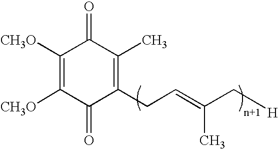
where n is equal to 9. CoQ₁₀ is typically found in an oxidized form (ubiquinone). However, CoQ₁₀ may be reduced to form reduced CoQ₁₀ (ubiquinol). References of CoQ₁₀ throughout the application may include the oxidized form, the reduced form, or combinations thereof.

The term “anti-microbial agent” refers to a substance that prevents the growth of disease-causing micro-organisms.

The term “dermatological health” refers to a positive state or the health condition of the skin.

As used herein, “pharmaceutically acceptable carrier” includes any material, which when combined with the conjugate retains the conjugates' activity and is non-reactive with the subject's immune system. Examples include, but are not limited to, any of the standard pharmaceutical carriers such as a phosphate buffered saline solution, water, emulsions such as oil/water emulsion, and various types of wetting agents. Other carriers may also include sterile solutions, tablets including coated tablets and capsules. Typically, such carriers contain excipients such as starch, milk, sugar, certain types of clay, gelatin, stearic acid or salts thereof, magnesium or calcium stearate, talc, vegetable fats or oils, gums, glycols, or other known excipients. Such carriers may also include flavor and color additives or other ingredients. Compositions comprising such carriers are formulated by well known conventional methods.

As used herein, “administering” means oral administration, administration as a suppository, topical contact, intravenous, intraperitoneal, intramuscular, intralesional, or subcutaneous administration, administration by inhalation, or the implantation of a slow-release device, e.g., a mini-osmotic pump, to the subject. Administration is by any route including parenteral and transmucosal (e.g., oral, nasal, vaginal, rectal, or transdermal), particularly by inhalation. Parenteral administration includes, e.g., intravenous, intramuscular, intra-arteriole, intradermal, subcutaneous, intraperitoneal, intraventricular, and intracranial. Moreover, where injection is to treat a tumor, e.g., induce apoptosis, administration may be directly to the tumor and/or into tissues surrounding the tumor. Other modes of delivery include, but are not limited to, the use of liposomal formulations, intravenous infusion, transdermal patches, etc.

The term “ameliorating” or “ameliorate” refers to any indicia of success in the treatment of a pathology or condition, including any objective or subjective parameter such as abatement, remission or diminishing of symptoms or an improvement in a patient's physical or mental well-being. Amelioration of symptoms can be based on objective or subjective parameters including the results of a physical examination and/or a psychiatric evaluation.

The term “therapy” refers to “treating” or “treatment” of a disease or condition including preventing the disease or condition from occurring in an animal that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease).

The term “effective amount” or “an amount effective to” or a “therapeutically effective amount” or any grammatically equivalent term means the amount that, when administered to an animal for treating a disease, is sufficient to effect treatment for that disease.

The term “anti-acne therapeutic” refers to a compound or method that effects the treatment or therapy of acne.

The term “enhanced” refers to any degree of betterment, augmentation embellishment, beautification, strengthening or improvement. Thus “enhanced performance” indicates that performance is improved in one state, by comparison to another.

The term “improved” refers to a more desirable condition than previously existed, or alternatively, improved refers to state wherein a more desirable result is achieved under one set of conditions as compared with another. Improvement is demonstrated by any indicia of success, betterment, progression, or amelioration including any objective or subjective parameter such as abatement, remission or diminishing of symptoms or an improvement in an individual's physical or mental well-being. Improvement can be based on objective or subjective parameters including the results of a physical examination and/or a psychiatric evaluation.

The term “sunscreening agent” refers to any composition or compound that prevents sunburn or otherwise minimizes skin damage due to exposure to the sun or other ultraviolet radiation.

The term “facial mask” refers to cosmetic preparation that is applied to the face used for cleansing and tightening the skin.

The term “glacial silt” refers to sedimentary material, derived from glaciers, comprised of very fine particles intermediate in size between sand and clay.

The term “cosmetic foundation” refers to a base layer of make-up which adds color and hides imperfections in one's complexion.

The term “skin lotion” refers to a suspension or emulsion for application to the skin.

The term “skin protection” refers to the prevention, safe-guarding, or shielding of the skin from harm or injury.

The term “isolated” refers to a material that is substantially or essentially free from components, which are used to produce the material. For compositions of the invention, the term “isolated” refers to material that is substantially or essentially free from components, which normally accompany the material in the mixture used to prepare the composition. “Isolated” and “pure” are used interchangeably. Typically, isolated components of the invention have a level of purity preferably expressed as a range. The lower end of the range of purity for the component is about 60%, about 70% or about 80% and the upper end of the range of purity is about 70%, about 80%, about 90% or more than about 90%.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multi-valent radicals, having the number of carbon atoms designated (i.e. C₁-C₁₀ means one to ten carbons). Examples of saturated hydrocarbon radicals include groups such as methyl, ethyl, n-propyl, isopropyl, n-butyl, t-butyl, isobutyl, sec-butyl, cyclohexyl, (cyclohexyl)ethyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below as “heteroalkyl,” “cycloalkyl” and “alkylene.” The term “alkylene” by itself or as part of another substituent means a divalent radical derived from an alkane, as exemplified by —CH₂CH₂CH₂CH₂—. Typically, an alkyl group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” refer to those groups having an alkyl group attached to the remainder of the molecule through an oxygen, nitrogen or sulfur atom, respectively. Similarly, the term “dialkylamino” is used in a conventional sense to refer to —NR′R″ wherein the R groups can be the same or different alkyl groups.

The term “acyl” or “alkanoyl” by itself or in combination with another term, means, unless otherwise stated, a stable straight or branched chain, or cyclic hydrocarbon radical, or combinations thereof, consisting of the stated number of carbon atoms and an acyl radical on at least one terminus of the alkane radical.

The term “heteroalkyl,” by itself or in combination with another term, means, unless otherwise stated, a stable straight or branched chain, or cyclic hydrocarbon radical, or combinations thereof, consisting of the stated number of carbon atoms and from one to three heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group. The heteroatom Si may be placed at any position of the heteroalkyl group, including the position at which the alkyl group is attached to the remainder of the molecule. Examples include —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Also included in the term “heteroalkyl” are those radicals described in more detail below as “heteroalkylene” and “heterocycloalkyl.” The term “heteroalkylene” by itself or as part of another substituent means a divalent radical derived from heteroalkyl, as exemplified by —CH₂—CH₂-S—CH₂CH₂— and —CH₂—S—CH₂—CH₂-NH—CH₂—. For heteroalkylene groups, heteroatoms can also occupy either or both of the chain termini. Still further, for alkylene and heteroalkylene linking groups, no orientation of the linking group is implied.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include cyclopentyl, cyclohexyl, 1-cyclohexenyl, 3-cyclohexenyl, cycloheptyl, and the like. Examples of heterocycloalkyl include 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “fluoroalkyl,” are meant to include monofluoroalkyl and polyfluoroalkyl.

The term “aryl,” employed alone or in combination with other terms (e.g., aryloxy, arylthioxy, arylalkyl) means, unless otherwise stated, an aromatic substituent which can be a single ring or multiple rings (up to three rings), which are fused together or linked covalently. “Heteroaryl” are those aryl groups having at least one heteroatom ring member. Typically, the rings each contain from zero to four heteroatoms selected from N, O, and S, wherein the nitrogen and sulfur atoms are optionally oxidized, and the nitrogen atom(s) are optionally quaternized. The “heteroaryl” groups can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl ring systems are selected from the group of acceptable substituents described below. The term “arylalkyl” is meant to include those radicals in which an aryl group is attached to an alkyl group (e.g., benzyl, phenethyl, pyridylmethyl and the like) or a heteroalkyl group (e.g., phenoxymethyl, 2-pyridyloxymethyl, 3-(1-naphthyloxy)propyl, and the like).

Each of the above terms (e.g., “alkyl,” “heteroalkyl” and “aryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be a variety of groups selected from, for example: —OR′, ═O, ═NR′, ═N—OR′, —NR′R″, —SR′, -halogen, —SiR′R″ R′″, —OC(O)R′, —C(O)R′, —CO₂R′, CONR′R″, —OC(O)NR′R″, —NR″C(O)R′, —NR′—C(O)NR″R′″, —NR″C(O)₂R′, —NH—C(NH₂)═NH, —NR′C(NH₂)═NH, —NH—C(NH₂)═NR′, —S(O)R′, —S(O)₂R′, —S(O)₂NR′R″, —CN and —NO₂ in a number ranging from zero to (2N+1), where N is the total number of carbon atoms in such radical. R′, R″ and R′″ each independently refer to hydrogen, unsubstituted (C₁-C₈) alkyl and heteroalkyl, unsubstituted aryl, aryl substituted with 1-3 halogens, unsubstituted alkyl, alkoxy or thioalkoxy groups, or aryl-(C₁-C₄)alkyl groups. When R′ and R″ are attached to the same nitrogen atom, they can be combined with the nitrogen atom to form a 5-, 6-, or 7-membered ring. For example, —NR′R″ is meant to include 1-pyrrolidinyl and 4-morpholinyl. From the above discussion of substituents, one of skill in the art will understand that the term “alkyl” is meant to include groups such as haloalkyl (e.g., —CF₃ and —CH₂CF₃) and acyl (e.g., —C(O)CH₃, —C(O)CF₃, —C(O)CH₂OCH₃, and the like).

Similarly, substituents for the aryl groups are varied and are selected from: -halogen, —OR′, —OC(O)R′, —NR′R″, —SR′, —R′, —CN, —NO₂, —CO₂R′, —CONR′R″, —C(O)R′, —OC(O)NR′ R″, —NR″C(O)R′, —NR″C(O)₂R′, —NR′—C(O)NR″R′″, —NH—C(NH₂)═NH, —NR′C(NH₂)═NH, —NH—C(NH₂)═NR′, —S(O)R′, —S(O)₂R′, —S(O)₂NR′R″, —N₃, —CH(Ph)₂, perfluoro(C₁-C₄)alkoxy, and perfluoro(C₁-C₄)alkyl, in a number ranging from zero to the total number of open valences on the aromatic ring system; and where R′, R″ and R′″ are independently selected from hydrogen, (C₁-C₈)alkyl and heteroalkyl, unsubstituted aryl, (unsubstituted aryl)-(C₁-C₄)alkyl, (unsubstituted aryl)oxy-(C₁-C₄)alkyl and perfluoro(C₁-C₄)alkyl.

As used herein, the term “heteroatom” is meant to include, for example, oxygen (O), nitrogen (N), sulfur (S) and silicon (Si).

Certain compounds of the present invention possess asymmetric carbon atoms (optical centers) or double bonds; the racemates, diastereomers, geometric isomers and individual isomers are all encompassed within the scope of the present invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

Introduction

The present invention provides formulations for enhanced and improved dermatological health. The inventions relate to the improved absorption of the pharmaceutical agents in conjunction with enhanced improvement of skin health by the inclusion of coenzyme Q₁₀ through new technological advancements for enhanced dispersity. The addition of coenzyme Q₁₀ results in new formulations that increase cellular health and minimize the effects of aging. As set forth herein, the invention also provides useful improvements in inter alia anti-acne formulations, skin protectant lotions, cosmetic foundations, and sunscreen formulations.

CoQ₁₀ may be combined with active molecules (e.g., camitine) and the hydrotrope, PTS. PTS is comprised of polyethylene glycol, tocopherol and sebacic acid; hence the name PTS. Active molecules may be water-soluble or lipophilic. The PTS converts the active molecule into a nanoparticle micelle, where the inner sphere is occupied by tocopherol, CoQ₁₀ and other active molecules. Together these become water-soluble. While this process may not be extremely helpful for already water-soluble active molecules, this process is very beneficial to lipophilic active molecule such as astaxanthin or cortisone. Therefore, PTS allows us to mix in lipophilic active or a water-soluble active molecule in creams, since creams use both phases in their formulation.

The Compounds

In a first aspect, the present invention provides compositions comprising a compound according to Formula (I):

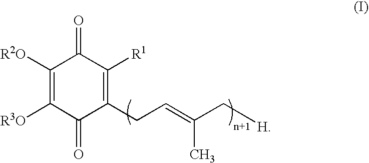

In Formula (I), R¹, R² and R³ are independently selected from substituted or unsubstituted C₁-C₆ alkyl groups and n is an integer from 0 to 19. In one embodiment, n is 9. In another embodiment, R¹, R² and R³ are methyl groups. In another embodiment, the compound is in a reduced form.

According to the invention, the compound according to Formula (I) is present in the composition at a weight percent of the total formulation in a range from about 0.5% to about 20%. In some embodiments the weight percent of the compound according to Formula (I) is present in a range from about 0.75% to about 15%, and in other embodiments the weight percent of the compound according to Formula (I) is present in a range from about 1.0% to about 10%. In still other embodiments, the weight percent of the compound according to Formula (I) is present in the composition at a weight percent of about 1%, 2%, 3%, 4%, 5%, 6%, 7%, 8%, 9%, 10%, 11%, 12%, 13%, 14%, 15%, 16%, 17%, 18%, 19%, or 20%.

In other aspects, the compositions of the invention further comprise a solubilizing agent according to Formula (II):

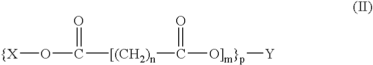

In Formula (II), X is a residue of a hydrophobic moiety, which is a member selected from sterols, tocopherols, and derivatives thereof; Y is a residue of a hydrophilic moiety, which is a member selected from polyethers, polyalcohols, and derivatives thereof; p is 1 or 2; m is 0 or 1; and n is an integer greater than or equal to 0. When p and m are equal to 1 and the hydrophobic moiety is cholesterol, n is greater than 4 and not equal to 8. When p and m are equal to 1, and the hydrophobic moiety is (+)-α-tocopherol, n is not equal to 2. In one embodiment, when the hydrophobic moiety is a member selected from cholesterol, 7-dehydrocholesterol, campesterol, sitosterol, ergosterol, stigmasterol, α-tocopherol, β-tocopherol, γ-tocopherol, and δ-tocopherol, the hydrophilic moiety is a polyether. In another embodiment, the polyether is a polyethylene glycol.

According to the invention, when present in a formulation, the solubilizing agent according to Formula (II) is present in the formulation at a concentration relative to the concentration of the compound according to Formula (I). In an exemplary embodiment, the solubilizing agent according to Formula (II) is present in the formulation at a ratio of solubilizing agent to coenzyme Q₁₀ of 2:1 mol/mol or 3:1 w/w.

Synthesis of the Compounds and Methods of the Invention

Techniques useful in synthesizing the compounds of the invention are both readily apparent and accessible to those of skill in the relevant art. In particular, cost-effective methods for synthesizing the ubiquinone compounds of the invention are disclosed in U.S. Pat. No. 6,852,895; U.S. Pat. No. 6,545,184; copending U.S. patent application Ser. No. 10/973,158, filed Oct. 24, 2004, and co-pending U.S. patent application Ser. No. 11/003,544, filed Dec. 3, 2004, each of which is incorporated herein by reference.

### The Embodiments

Acne Medications

In one embodiment, the invention provides compositions comprising a compound according to Formula (I) for the treatment of acne, and for improved dermatological health. In one embodiment, adapalene is the active ingredient present in a formulation ranging from about 0.01% to about 5% per gram. In some embodiments, adapalene is present at a concentration of about 0.1% per gram provided in the form of a topical gel delivery system.

Adapalene is effective for the treatment of existing acne lesions and to prevent the development or further proliferation. The medication can be used for an indefinite period of time with minimal side effects. Adapalene is a chemically stable, retinoid-like analog containing a carboxylic acid residue. Unfortunately, it is only sparingly soluble in polar solvents such as ethanol, and is virtually insoluble in water. For adapalene to be effective, it must be absorbed through epithelial skin cells of which there are multiple layers. Due to its natural chemical makeup, it is unable to achieve even the lowest level of absorption and thus cannot be sold as a single entity product. Even when combined with other fat and water soluble ingredients, absorption of adapalene is still relatively low (<0.25 ng/mL) after chronic topical use as measured in controlled clinical studies. The invention provides formulations of adapalene that increase absorption and thereby improve performance of the medication. Furthermore, the inclusion of a compound according to Formula (I) in the composition also lessens the occurrence of side effects and contraindications.

In one embodiment, increased absorption is reflected as a requirement for less medication to be administered for the same effectiveness as that of a product not containing a compound according to Formula (I). In other embodiments, increased absorption results in enhanced results in a significantly shorter period of time.

Without being bound by theory, it is thought that the ability of CoQ₁₀ to increase absorption of adapalene is achieved through its highly lipophilic makeup. Absorption may be further increased by recent technological developments disclosed in U.S. Pat. No. 6,191,172 (incorporated herein by reference) that allow for dramatically increased dispersity and bioavailability.

In another embodiment, azelaic acid, a naturally occurring saturated dicarboxylic acid, is used in combination with a compound according to Formula (I). Azelaic acid may be present in the formulation in amounts ranging from about <1%, 5%, 7%, 10%, 12%, 15%, 16%, 17%, 18%, 19%, 20%, 25%, 30%, 35% or more.

In vitro research has shown that azelaic acid also possesses antimicrobial activity, as well as normalization of keratinization. Up to 10% of the applied dose to human skin (in vitro) was found in the epidermis and dermis. Human clinical trials have shown azelaic acid is effective in helping to eliminate mild-to-moderate inflammatory acne, although the exact mechanism of action is not known. Side effects appear to be mild, with a half-life of 12 hours after application suggesting that absorption of azelaic acid is limited. Fortunately, absorption of azelaic acid is increased by the compositions of the invention providing formulations permitting the same results with less medication. In other cases the formulations of the invention provide enhanced results in a shorter period of time.

In another embodiment, the invention provides formulations for the treatment of acne, and for improved dermatological health that comprise a compound according to Formula (I) and a retinoid compound. In one such embodiment, treatment of nodular/cystic acne is effected by 13-cis-retinoic acid, in combination with a compound according to Formula (I). 13-cis-retinoic acid is not currently available as a topical treatment due its low absorption rate and rapid degradation. Oral administration is well-known to often give rise to side effects that can be severe and debilitating. Thus, this type of treatment is almost always a last resort. Prescriptions for 13-cis-retinoic acid are made only when the skin is severely damaged with little hope of full aesthetic recovery.

However, a novel formulation for topically delivering retinoic acid and therapeutically effective salts thereof is available and offers significant advantages over preexisting formulations (e.g., U.S. Pat. No. 5,955,109). Retinoic acid (Formula (III)) and 13-cis-retinoic acid (Formula (IV)), the structures of which are shown below, are geometric isomers and show reversible interconversion. 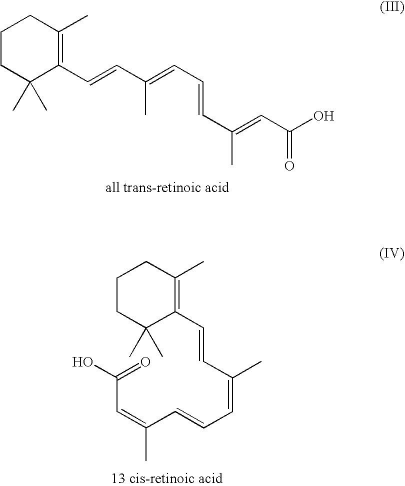
Administration of one isomer will give rise to the other. Topically administered retinoic acid uses the patented bead system to achieve the same effectiveness of acne treatment with retinoic acid as can be achieved with 13-cis-retinoic acid. Retinol (vitamin A) may also be combined with Formula (I) for the treatment of acne.

An improved formulation comes about by the inclusion of a compound of Formula (I), in particular, by the inclusion of CoQ₁₀. CoQ₁₀ is produced endogenously and found in every cell of the human body. CoQ₁₀ plays an essential role in the mitochondrial electron transport chain and the production of biochemical energy. Additionally, L-carnitine aids in energy production in cells by transporting fatty units across the mitochondria membrane to allow for oxidation of fats. L-carnitine may also include ester derivatives (e.g., acetyl carnitine, etc.). Furthermore, CoQ₁₀'s role as an antioxidant neutralizes potentially damaging free radicals created in part by the energy-generating process. Without being bound by theory it is thought that CoQ₁₀'s ability to quench free radicals works in conjunction with retinoic acid by improving the skin's overall appearance and cellular health. As with other formulations of the invention, CoQ₁₀ can increases absorption of retinoic acid through its highly lipophilic makeup.

Protectant Ointments

In another embodiment, the invention provides ointments comprising a compound according to Formula (I). Ointments serve a multitude of dermatological purposes. The chief purpose of ointments is their role as protectants of skin during the healing process, in particular for skin that has been badly irritated (e.g., by a burn) or that has become infected. For such conditions, antimicrobial soaps and hand disinfectants are plentiful; however, far more effective are antibiotic ointments. Regulated by the government as over-the-counter (“OTC”) medicines, they can be counted on to provide documented levels of protection against further infection. These ointments are routinely sold in most food and drug stores.

The addition of coenzyme Q₁₀ to ointments, represents an unprecedented formulation which enhances the healing properties of these salves. Without being bound by theory, it is thought that enhanced healing properties could be brought about by protecting damaged cells from additional attack by free radicals (i.e., as an antioxidant). Enhanced healing means that the healing occurs about 5% faster, 6% faster, 7% faster, 10% faster, 20% faster, 25% faster, 30% faster, 40% faster, 100% faster, 200% faster, 1000% faster or more than with ointments that do not contain a compound according to Formula (I). The very nature of endogenously produced CoQ₁₀ makes it an ideal additive to the lipophilic environment of an ointment, as its penetration into the skin would lead to improved energy levels within the affected area thereby increasing the rate of recovery. Furthermore, the penetration of CoQ₁₀ facilitates the penetration of the antibiotic component into the skin, thereby providing increased absorption of the antibiotic. In one embodiment the ointment also comprises an analgesic e.g., capsaicin, or salicylic acid. In another embodiment, the ointment further comprises an anti-inflammatory agent (e.g., fluticasone propionate). In another embodiment, the anti-inflammatory agent comprises cortisone. Cortisone is a type of steroid produced naturally by the adrenal gland of the human body and is used to treat inflammation and dermititis.

Sunscreens

More than 1,000,000 new cases of skin cancer are diagnosed yearly in the United States. Sunscreen comes in many forms; lotion, oil, stick, gel and cream. The sole purpose until recently was simply to block out the damaging UV rays. However, recently more ingredients have been added to improve skin health and decrease the appearance of aging. Collagen, elastin, vitamins and botanicals can be found in various sunscreen products. The addition of coenzyme Q₁₀ would enhance the ability of sunscreen to protect the cells. Therefore, in another embodiment, the invention provides improved sunscreen formulations comprising a compound according to Formula (I). In some embodiments, the formulation may further comprises a compound according to Formula (II), which results in a considerably greater level of bioavailability of CoQ₁₀. The presence of highly dispersed, more bioavailable CoQ₁₀ in sunscreens as disclosed in this application represents an as yet unprecedented formulation.

The effectiveness of a sunscreen formulation is often reported as a sun protection factor (“SPF”). SPF stipulates the amount of protection a sunscreen should provide from ultraviolet B (“UVB”) radiation, which is the main cause of sunburn. Skin damage from sunlight builds up with continued exposure, whether sunburn occurs or not. In addition to skin cancer and sunburn, other effects include wrinkling, premature aging, and in time, an almost leathery appearance of the skin. Research also suggests that excessive exposure to UV radiation may interfere with the body's immune system.

Although sunburn is caused by the shorter ultraviolet wavelengths, known as UVB, the longer wavelengths, known as ultraviolet A (“UVA”), can penetrate the skin to a much deeper level and damage connective tissue. It is important to limit exposure to both UVA and UVB rays.

Sunscreens prevent the formation of squamous cell carcinomas in animals. In humans, the regular use of sunscreens has been shown to reduce actinic keratoses, solar elastosis and squamous cell carcinoma. Drug photosensitization and photo-induced or photo-aggravated dermatoses may be avoided with sunscreen use, especially with products that offer better blockage in the UVA range.

The SPF is defined as the UV energy required to produce 1 minimal erythema dose (“MED”) on protected skin divided by the UV energy required to produce a MED on unprotected skin, which may also be defined by the following ratio: SPF value=MED (protected skin (“PS”))/MED (unprotected skin (“US”)), where MED (PS) is the minimal erythema dose for protected skin after application of 2 mg/cm of the product, and MED (US) is the minimal erythema dose for unprotected skin, i.e., skin to which no sunscreen product has been applied. In effect, the SPF value is the reciprocal of the effective transmission of the product viewed as a UV radiation filter.

The sunscreen formulations of the invention increase the SPF of the sunscreen by about 1%, 2%, 3%, 4%, 5%, 10%, 15%, 20%, 50%, 100%, 200%, 1000% or more over that of sunscreen formulations that do not contain a compound according to Formula (I).

Alternatively, the effectiveness of a sunscreen is measured as a UVA protection value by estimating the reduction of UVA radiation brought about by a sunscreen preparation. An exactly defined test procedure using a photometric technique is employed; for example, the Australian Standard 2604:93. The result of such tests is a UVA protection value, expressed as a percentage. Protection products that meet this strict standard must absorb at least 90% of the UVA radiation.

Cosmetics

Lip Balm and Lipstick

In one embodiment, the invention provides for a lipstick formulation comprising a compound according to Formula (I). In some embodiments, the lipstick formulation also comprises a sunscreening agent. In an exemplary embodiment, the compound according to Formula (I) is coenzyme Q₁₀.

The cosmetics industry offers numerous formulations of lipstick. They vary by color, method of application, shine level, and duration on the lips. Only within the past few years have value added ingredients such as moisturizers been appreciated and added to lipstick. In contrast to other cosmetics, lipstick is often applied countless times throughout the day. Of the numerous forms of lipstick, the new matte lipstick imparts greater color and is longer lasting, but unfortunately it can dry the lips in just a single application. Many women now choose lipstick by the quality of value-added ingredients found in these products, rather than by their favorite brand name alone.

Coenzyme Q₁₀ is an attractive added ingredient to any lipstick formulation. It is produced endogenously in all cells and, therefore, is already present in the skin cells in lips where it plays an essential role in the mitochondrial electron transport chain and the production of biochemical energy. Additionally, L-carnitine aids in energy production in cells by transporting fatty units across the mitochondria membrane to allow for oxidation of fats. L-carnitine may also include ester derivatives (e.g., acetyl camitine, etc.). As people age, the levels of CoQ₁₀ in all cells drop, and therefore, supplementation is essential. The presence of CoQ₁₀ in lipstick readily benefits the lips by augmenting natural levels supplying a potent antioxidant to neutralize potentially damaging free radicals created in part by the body's natural energy-generating processes.

With its highly lipophilic characteristics together with recent technological developments that allow for dramatically increased dispersity and bioavailability, CoQ₁₀'s ability to quench free radicals at the cellular level can aid in the beauty and long-term health of the lips. Furthermore, cosmetic products comprising CoQ₁₀ provide increased protection from the sun's damaging UV rays.

In another embodiment, the invention provides formulations for lip balm which protects the lips from the elements and also contributes to enhanced dermatological health of the skin of the lips.

Lip balm was developed as a way to protect the delicate tissue of the lips. Until recently, it was waxy and offered only protection by coating the lips. Lip balm has since improved and now provides protection through various sunscreens and antioxidants, as well as vitamins and botanical extracts to moisturize, natural fruit extracts for flavoring, and medicinal ingredients to enhance healing for severely chapped lips.

New to lip balm are ingredients added to beautify the lips by imparting color, enhancing lip tone and decreasing the appearance of characteristics associated with aging. An ideal addition to the current lip balm in the marketplace is coenzyme Q₁₀. It is produced endogenously in all cells and, therefore, is already present in the skin cells in lips where it plays an essential role in the mitochondrial electron transport chain and thus, the production of biochemical energy. Additionally, L-carnitine aids in energy production in cells by transporting fatty units across the mitochondria membrane to allow for oxidation of fats. L-carnitine may also include ester derivatives (e.g., acetyl carnitine, etc.).

As people age, the levels of CoQ₁₀ in all cells drop, and thus, supplementation is essential. Thus, the presence of CoQ₁₀ in lip balm readily benefits the lips by augmenting natural levels, and thereby supplying a potent antioxidant to neutralize potentially damaging free radicals created in part by the body's natural energy-generating processes. With its highly lipophilic characteristics together with recent technological developments that allow for dramatically increased dispersity, the ability of CoQ₁₀ to quench free radicals at the cellular level can aid in the beauty and long-term health of the lips.

Cosmetic Foundation

In another exemplary embodiment, the invention provides a formulation for a cosmetic foundation. In an exemplary embodiment, the cosmetic foundation comprises a compound according to Formula (I). In a particular exemplary embodiment, the compound according to Formula (I) is coenzyme Q₁₀.

Applying foundation is typically the first step in a woman's makeup routine. Foundation adds color and hides imperfections in one's complexion. Foundation is the one truly essential element to a woman's makeup regimen. One important characteristic of a foundation is its ease of application. Better foundation formulations spread onto the skin uniformly and adhere evenly to the skin for extended periods of time. Added ingredients must either enhance or cause no interference to the color of the foundation, its applicability, or its ability to adhere to the skin.

Cosmetic companies continue to develop new formulations with value-added ingredients. Their roles are to minimize damaging oxidation caused by environmental factors, diet, and natural metabolic processes so as to improve overall skin health, improve skin tone, decrease the appearance of fine lines, moisturize, and protect against the sun's damaging UV light. Naturally occurring additives, as opposed to synthetic, are best tolerated. Coenzyme Q₁₀ is an exceptional value-added ingredient. Surprisingly however, prior to the present invention cosmetic foundations were not formulated to comprise this ingredient. Coenzyme Q₁₀ is produced endogenously and found in every cell in the human body. Unlike many additional ingredients that are currently present in foundation, CoQ₁₀ has been shown to provide significant benefits to skin cells upon absorption, and furthermore facilitates the spreadability and applicability of the cosmetic foundation. Furthermore, CoQ₁₀ enhances the SPF factor of cosmetic foundations that comprise a sunscreening agent.

Cleansers

In another exemplary embodiment, the invention provides a composition for cleansing. The composition comprises the compound of Formula (I) in combination with a solubilizing agent of Formula (II).

Soap formulations of the invention avoid stripping the skin of its natural protective barrier, and also enhance the skin's appearance. Soaps and cleansers of the invention make possible healthier skin cells that hold onto moisture and thereby decrease visible fine lines.

Coenzyme Q₁₀ is produced endogenously and found in every cell in the human body. Unlike most additional ingredients that are currently present in soap, CoQ₁₀ has been shown to provide significant benefits to skin cells through its ability to quench free radicals. Further, with its highly lipophilic characteristics CoQ₁₀ facilitates the cleaning action of soaps and facial cleansers. Thus, the invention provides an ideal soap.

CoQ₁₀ can easily be formulated into bar soap in combination with a solubilizing agent such as that of Formula (II) thereby providing a formula that improves the cleansing action of the soap, and provides for increased bioavailability of this important bionutrient. Methods for producing soaps are known in the art (e.g., U.S. Pat. Nos. 6,794,344; 6,846,785; 6,809,071; 6,706,675; and 6,325,882).

Masks

Clay masks are commonly used for maintaining a healthy complexion. Once the skin's surface has been gently cleansed to remove dirt, grime, and impurities, masks serve as mineral-enriched treatments that moisturize, detoxify, and exfoliate the skin, leaving it soft and smooth. Masks can also help to unclog pores, improve pH balance, and minimize the appearance of lines and wrinkles due to aging. Glacial silt functions as an especially fine, natural abrasive for cleansing the skin's surface. Coenzyme Q₁₀, a key constituent of all human cells responsible for production of cellular energy, can be formulated into the facial mask to enhance the quality of the cleansing process through absorption into the skin. Being far more lipophilic than other vitamins frequently found in facial cleansers (e.g., vitamins C and E), CoQ₁₀ is more highly absorbed thereby improving the health and therefore the appearance of the skin.

Lotions

Formulation with Hyaluronic Acid

In another embodiment, the invention provides a formulation comprising a compound of Formula (I), and hyaluronic acid. In one embodiment, the compound of Formula (I) is coenzyme Q₁₀. In another embodiment, the formulation is a skin lotion.

Hyaluronic acid (HA) is a glycosaminoglycan comprised of D-glucuronic acid and N-acetyl glucosamine. Produced within the body, it is a compound of high molecular weight. Its weight lends itself to high viscosity and excellent lubrication of skin and joints. It is a major constituent of the extracellular matrix. The components of the matrix are produced within the cells and then secreted to the extracellular space. HA is found in large amounts in the vitreous humor of the eye, in the synovial fluid and the cartilage. However the largest single portion, amounting to fifty percent of the HA in the body, can be found in the skin located throughout the extracellular matrix and the connective tissue. Clinical research has indicated that as people age, HA shifts within skin cells moving from the upper layers of the epidermis to the lower layers of the dermis. As the role of HA is to act as a sponge and hold onto moisture in the skin, this shift away from the top layers causes an increase in fine lines and a decrease in skin elasticity.

Topical HA, if absorbed into the top layers of the epidermis, would increase the moisture content of the skin and decrease the appearance of fine lines while increasing lost elasticity. Unfortunately, when used by itself, absorption of HA is hampered by its high viscosity. For HA to be effectively delivered in a topical application, it must be combined with another ingredient that acts as a carrier. High bioavailability coenzyme Q₁₀ is an ideal carrier, which also adds its own invaluable benefits to the skin. Therefore, the invention provides a formulation for a skin lotion that combines HA with CoQ₁₀ to provide superior absorption of HA into the upper layers of the dermis, and increased cellular energy due to the presence of CoQ₁₀.

Formulation with Antioxidants

Sun, stress, pollution, smoking, and unhealthy eating and drinking habits are often cited as the primary causes of wrinkles in skin, rather than the simple notion that these come with age. To combat aging skin, a wealth of products has been brought to the market. Chief among these products is retinoic acid (“retin A”) which is a common additive. Unfortunately, retinoic acid can cause heightened sun sensitivity, and is therefore less than desirable as an additive in an anti-wrinkle formulation.

Advances in skin care products have led to alternatives that offer advantages due to added antioxidants, such as vitamin A, vitamin E, astaxanthin, alpha-lipoic acid, zeaxanthin, lutein, flavonoids (e.g. from natural sources, fruits, vegetables, berries, etc.) and pycnogenol. Astaxanthin is a naturally carotenoid pigment that maximizes the body's use of carotenoids and vitamin E. Alpha-lipoic acid protects the mitochondria and DNA and works closely with other anti-oxidants by recycling them and making them more effective. Most recently, advances in skin care products have focused on vitamin C working in tandem with minerals (e.g., zinc) and amino acids (e.g., tyrosine). Although anti-wrinkle results with such creams have been impressive, the half life of vitamin C is far too short (i.e., 30 minutes) to anticipate bioactivity over time due to autoxidation of this key ingredient. An antioxidant of equal or better potency than vitamin C, is coenzyme Q₁₀. The half life of CoQ₁₀ (>30 h) is far greater than that of vitamin C. Moreover, being highly lipophilic, it is more readily absorbed than vitamin C and hence, more protective of the skin towards damaging free radicals. Therefore, in one embodiment, the invention provides a new formulation for an anti-wrinkle skin lotion that combines antioxidants with highly bioavailable CoQ₁₀, together with the appropriate minerals and amino acids. Ester derivatives may also be included when the parent compounds vitamin A, vitamin C, vitamin D and vitamin E are discussed.

Formulations with Petroleum Products

In another embodiment, the invention provides a composition for skin protection comprising a compound of Formula (I), and a petroleum product, e.g., petroleum jelly or mineral oil. Petroleum jelly when applied to the skin, provides a barrier to the harsh environment. It has the ability to protect the integrity of the skin and ward off infection.

Because of its chemical structure, little of the petroleum jelly gets absorbed into the epidermis. The oily nature of petroleum jelly makes it ideally suited to admixture with coenzyme Q₁₀ with its natural lipophilic characteristics. Therefore, the invention provides formulations of petroleum jelly with CoQ₁₀ to improve the overall health of the skin cells by providing CoQ₁₀ for absorption into the upper skin layers. Thus, the composition enhances the function of petroleum jelly.

Mineral oil seals moisture into the skin, and provides a barrier to protect delicate skin. For ease of use, the oil can be misted on to the skin. CoQ₁₀ with its natural lipophilic characteristics mixes well with oily substances such as mineral oil.

Mineral oil compositions comprising CoQ₁₀ are highly beneficial. When applied to the skin, the mineral oil positions the CoQ₁₀ to penetrate the skin that is being treated. In one embodiment, the mineral oil composition comprising CoQ₁₀ is applied to skin to protect the skin from chapping. In another embodiment, the mineral oil composition comprising CoQ₁₀ is applied to skin to facilitate healing of diaper rash.

In another embodiment, the mineral oil formulation comprising CoQ₁₀ is comprised within diapers. Thus, the diapers comprising the mineral oil formulation of the invention function as a giant transdermal patch to deliver CoQ₁₀ to the skin for the healing or prevention of diaper rash. Further, in some embodiments, CoQ₁₀ helps to deliver antifungal agents that are also comprised within the linings of babies diapers.

In other embodiments, recent technological developments that provide for dramatically increased dispersity of CoQ₁₀ (e.g., U.S. Pat. No. 6,191,172), improve the absorption of CoQ₁₀ from the mineral oil formulation into the upper skin layers, and the improved absorption of CoQ₁₀ further improves the overall health of skin cells.

In still further embodiments, CoQ₁₀ compositions comprising CoQ₁₀ and natural vegetable oils such as olive oil, corn oil, and the like are formulated to provide lotions that, when applied topically to the skin, improve skin health and appearance.

Transdermal Delivery

In another exemplary embodiment, the invention provides a method and device for transdermal delivery of a composition comprising a compound according to Formula (I). In one embodiment, the transdermal delivery system is a patch.

Many formats are known in the art to effect transdermal delivery of agents. In an exemplary embodiment, the transdermal delivery system is a transdermal patch or adhesive patch. Transdermal patches are useful for the delivery of controlled doses of a drug through the skin over a period of time. As is known in the art, a skin patch uses a special membrane to control the rate at which the liquid drug contained in the reservoir within the patch can pass through the skin and into the bloodstream. An example of a patch type transdermal delivery system is disclosed in U.S. Pat. No. 6,183,770 which is incorporated herein by reference. According to the invention, coenzyme Q₁₀ can be delivered alone via a transdermal skin patch. In alternative embodiments, CoQ₁₀ is delivered in combination with other substances, thereby improving the transdermal delivery of the substances with which it is combined, and providing a skin-enhancing material.

Delivery of agents to the skin for local skin treatment may be desirable to improve the health and appearance of the skin. Skin conditions such as dry or oily skin, blemishes, abrasions, cuts or rashes, for example, require localized skin treatment to remedy the affected area and to prevent further skin damage. If skin conditions are not treated, further skin irritation may occur resulting in infections or damage to the skin. Thus, treatment of skin conditions improves the health and appearance of the skin.

Further, the skin provides an ideal system for the delivery of agents to be active systemically. Although the skin is thought to provide the body with a protective covering, as is known in the art, a number of agents are absorbed through the skin, and since the skin is a highly vascularized organ, the agents may also penetrate into the circulating blood supply for distribution throughout the body, ultimately reaching the intended targets. Transdermal medication delivery provides constant and continuous absorption of the drug, thus keeping blood levels containing a drug within the therapeutic window. In addition, through transdermal delivery, medications may be introduced into the circulatory system without initially entering the portal circulation where they may be metabolized into a pharmacologically inactive form.

Thus, a patch that utilizes coenzyme Q₁₀ as the time-release carrier for agents across the skin, and potentially into the circulatory system, provides a valued nutrient into the skin while ensuring efficient delivery of a therapeutic substance. For example, drugs administered through skin patches in combination with coenzyme Q₁₀ include, but are not limited to retinoic acid, azelex, adapalene (for acne treatment), scopolamine (for motion sickness), nicotine (for smoking cessation), estrogen (for birth control, menopause, and to prevent osteoporosis after menopause), nitroglycerin (for angina), and lidocaine to relieve the pain of shingles.

In another exemplary embodiment, transdermal delivery is effected by transdermal delivery though the oral mucosa. Oral mucosa has more lipophilic cells than other mucosae, allowing for the delivery of lipophilic medications. Thus, in one embodiment, CoQ₁₀ is delivered alone or in combination with other substances across the oral mucosa.

The following examples are provided to illustrate the compositions and methods of the present invention, but not to limit the claimed invention.

### EXAMPLES

### Example 1

Anti-acne Agent—Lipid Formulations

The below formulations represent some typical anti-acne formulations according to the invention. When these formulations are topically applied to the skin, the symptoms of acne are improved.

| | Concentration (weight %) | | | |
|---|---|---|---|---|
| | | | | Ranges |
| Components | Ex 1 | Ex 2 | Ex 3 | Ex 4 |
| CoQ10 | 1 | 1 | 1 | 1 |
| Retinol | 0.05 | 0 | 0.05 | 0-2 |
| Benzoyl Peroxide* | 0 | 2 | 2 | 0-10 |
| Elubiol** | 0.1 | 0.1 | 0.1 | 0.001-2 |
| Salicylic acid*** | 2 | 0 | 0 | 0-10 |
| Ethanol | 60 | 40 | 20 | 20-80 |
| 1,3-butylene glycol | 12.5 | 20 | 20 | 5-20 |
| Propylene Glycol | 12.5 | 20 | 20 | 1-40 |
| Glyceryl Distearate | 2 | 0 | 0 | 0.0-5 |
| Cholesterol | 0 | 0 | 0.5 | 0-1 |
| Glyceryl Dilaurate | 0 | 0 | 1 | 0-5 |
| Laureth-9 | 2 | 0 | 1 | 0-5 |
| Polyoxyethylene- | 1 | 5 | 2.5 | 0-5 |
| 10-stearyl ether | | | | |
| (Brig 76) | | | | |
| Fragrance | 0.25 | 0.25 | 0.25 | 0-0.5 |
| Polyacrylamine, Laueth-7, | 0 | 0 | 3 | 0-5 |
| and C13-14 Isoparaffin | | | | |
| Sepigel 305) | | | | |
| Polyoxypropylene- | 0 | 5 | 0 | 1-20 |
| polyoxyethylene block | | | | |
| polymers | | | | |
| (Pluronic F-127) | | | | |
| Water | Q.S. to | Q.S. to | Q.S. to | Q.S. to |
| | 100% | 100% | 100% | 100% |

*available from Alzo Inc., Matawan, NJ
**available from Janssen Pharmaceutica N.V., Beerse, Belgium under the trade name, “Elubiol”;
***available from NIPA Laboratories Inc., Great Britain, UK

In one embodiment, the topical composition, comprises azelaic acid, as disclosed below. In one embodiment, the formulation is a cream formulation. Typically, the cream formulation may comprise:

Formulation A

| Ingredients | Weight Percent |
|---|---|
| CoQ10 | 1.00 |
| Azelaic Acid | 20.00 |
| Benzoic Acid DAB | 0.20 |
| Propylene Glycol USP | 12.50 |
| Cuntina CBS(1) | 7.00 |
| PEG-5 Glyceryl Stearate(2) | 5.00 |
| Cetearyl Octanoate(3) | 3.00 |
| Glycerin (85%) DAB | 1.5 |
| Purified Water DAB(4) | QS |

(1)Glyceryl Stearate (and) Cetearyl Alcohol (and) Cetyl Palmitate (and) Cocoglycerides (HENKEL)
(2)Arlatone percent (w/w)

### Example 2

Cosmetic Foundation Formulations

2.1 Oil-Emulsion Liquid Formulation

A cosmetic foundation formulation composition is made according to the following formula:

| | w/w % |
|---|---|
| CoQ10 | 1.00 |
| Cyclomethicone | 3.00 |
| Propyl paraben/laureth-7 | 0.75 |
| Mica/methicone | 0.01 |
| Red iron oxide/methicone | 2.70 |
| Yellow iron oxide/methicone | 2.70 |
| Black iron oxide/methicone | 2.70 |
| Titanium dioxide/cyclomethicone/ | 14.10 |
| dimethicone copolyol | |
| Zinc oxide/cyclomethicone/dimethicone | 5.00 |
| copolyol | |
| Cyclomethicone/Titanium dioxide/dimethicone | 3.80 |
| copolyol/triethoxy caprylyl silane | |
| Spherical silica | 0.15 |
| Nylon-12 | 1.00 |
| Boron nitride | 1.05 |
| Titanium dioxide/methicone | 1.00 |
| Dimethicone | 7.25 |
| Cyclomethicone | 5.80 |
| Tribehenin | 0.10 |
| Retinyl palmitate | 0.01 |
| Tocopheryl acetate | 0.01 |
| Aloe extract | 0.01 |
| Dimethicone | 1.50 |
| Polyglyceryl-4-isostearate | 1.50 |
| Cyclomethicone/dimethicone | 3.40 |
| Water | 31.05 |
| Salicylic acid/hydrolyzed vegetable protein | 0.50 |
| Methoxypropylgluconamide | 0.50 |
| Magnesium ascorbyl phosphate | 0.01 |
| Ethyl paraben/propylene glycol | 5.75 |
| Propylene glycol | 2.37 |
| Tetrasodium EDTA | 0.01 |
| Magnesium sulfate | 0.01 |
| Chamomile extract | 0.01 |
| Phytoclar | 0.01 |
| Soy protein | 3.00 |
| Cyclomethicone/dimethiconol | 2.00 |
| Methyl dihydrojasmonate | 0.25 |

The composition is prepared by combining the ingredients and mixing well to form an emulsion. The resulting makeup composition is poured into containers.

2.2 Oil-in-Water Emulsion Stick Foundation

An oil-in water Emulsion stick foundation formulation is made according to the following formula:

| | w/w % |
|---|---|
| CoQ10 | 1.00 |
| Dimethicone | 12.44 |
| Titanium dioxide | 4.80 |
| Polyglyceryl-6- | 0.39 |
| polyricinoleate | |
| Aluminum stearate | 0.62 |
| Cyclomethicone | 3.51 |
| Propyl paraben | 0.10 |
| Iron oxide yellow* | 1.00 |
| Iron oxide red* | 0.20 |
| Iron oxide black* | 0.08 |
| Talc | 0.85 |
| Nylon-12 | 0.25 |
| Synthetic wax | 1.50 |
| Isostearyl alcohol | 5.70 |
| Hydrogenated castor | 1.50 |
| oil | |
| Water | 41.03 |
| Ascorbic acid | 0.10 |
| Sodium stearate | 7.55 |
| Butylene glycol | 13.00 |
| Methyl paraben | 0.30 |
| PEG-20 methyl | 3.49 |
| glycosesquiisostearate | |
| PPC** | 0.86 |
| 10% calcium chloride | 0.23 |
| solution | |
| Phenoxyethanol | 0.50 |
| Tocopheryl acetate | 0.10 |
| Retinyl palmitate | 0.10 |
| Ethylene brassylate | 0.15 |

*pigments coated with perfluoropolymethyl isopropyl ether.
**the PPC was a complex of casein and carageenan in a weight ratio of about 20 percent casein and 80 percent carageenan by weight of the total PPC

The pigment grind is made by mixing, in an open colloid mill Sequences 1, 2, 3, and 4 and the dispersion is checked between two glass slides. Sequence 5 ingredients were then added and the mixture heated to 85° C. to 87° C. using a propellar mixer until the mixture is smooth and uniform. In a separate container, Sequence 6 is heated to 90° C. Sequences 7 through 12 were added to Sequence 6 using a propellar mixer while maintaining the temperature from 85° C. to 90° C. until the ingredients were dissolved and a milky white solution is obtained. The mixture of Sequences 1 through 4 is added and allowed to mix for 10 minutes with moderate agitation. Sequence 13 is added while maintaining the temperature at 83° C. to 85° C. and mixing for 5 minutes. Sequences 14, 15, and 17 were added to the mixture and mixed well. The composition is poured into stick molds.

### Example 3

Sunscreen Compositions and Measurement of SPF

3.1 Measurement of SPF

MED is the measurement of the amount of UVB radiation that is needed to induce a barely noticeable reddening (erythema). The SPF can be calculated using the following formula:

$$• = SPF = \frac{MED on protected skin}{MED on unprotected skin} = \frac{MED ( PS )}{MED ( US )}$$

A test begins by determining the individual UV sensitivity of the test participant by exposing an unprotected skin area on the back. The radiation sources are solar simulators, most equipped with xenon lamps; their spectrum is sun-like but of a higher intensity and therefore a shorter radiation period is possible. Approximately 20 hours after exposure to varying intensities, the MED (US) is determined by visual assessment of the erythemas in six skin areas.

For the actual SPF determination, test areas are marked out on the participant's back for the application of the sunscreen preparation. Every product area has a neighboring untreated control area assigned to it. The intensity of the exposure is determined according to skin sensitivity and the expected sun protection factor. After approximately 20 hours, the MED (US) and MED (PS) are visually assessed. The resulting sun protection factor gives the average increase in the individual time periods needed to produce an erythema after the application of the sun-screen.

3.1a SPF. COLIPA Method

The COLIPA method is an exemplary method used to determine the sun protection factor (UVB protection) of sunscreens (see e.g., COLIPA Sun Protection Factor Test Method, The European Cosmetic, Toiletry, and Perfumery Association, Brussels, Belgium October, 1994.). The test method is standardized and regulated. The radiation range and the output of the sun simulator used for testing are defined exactly. The amount applied and mode of product application are also laid down precisely. The test method is independent of skin type or the age of the test subjects. These precise specifications mean that statistically significant sunscreen testing can take place with only ten volunteers. The COLIPA method is a method that yields reproducible results with a high degree of reliability.

3.2 Sunscreen Formulations

This formulation is used to create a cosmetic foundation that provide water-resistant protection against the erythema-causing radiation of sunlight.

3.2a Water-in-oil Emulsion

Water-in-oil Emulsion with Isooctyl Acrylate: Stearyl Methacrylate: Acrylic Acid Terpolymer (mole ratio 50:30:20)

A one quart amber bottle is charged with 360 parts isopropyl palmitate (IPP), (“Emerest” 2316, Malmstrom Chemicals, Emery Industries, Inc.), 106.25 parts isooctyl acrylate, 117.12 parts stearyl methacrylate, 16.63 parts acrylic acid and 2.40 parts 70 percent benzoyl peroxide (“Lucidol” 70, Lucidol Division, Penwalt Corporation). The system is degassed by pulling a vacuum and releasing the latter with nitrogen. The bottle is subsequently capped and placed in an Atlas Launder-ometer at 60° C. for 16 hours. The clear viscous polymer is allowed to cool. A diluted sample of the polymer (2 parts polymer mixture with 7 parts IPP) gave a Brookfield viscosity (Spindle #3, 30 rpm) of 5400 cps.

The terpolymer as diluted above is incorporated into a sunscreening composition of the water-in-oil emulsion type containing the following ingredients.

| | Parts by |
|---|---|
| Ingredient | Weight |
| Phase A | |
| CoQ10 | 1 |
| Terpolymer solution | 9 |
| C.sub.11-C.sub.13 isoparaffinic solvent | 7 |
| (“Isopar” L, Exxon Corp.) | |
| C.sub.12-C.sub.15 alcohols | 3 |
| (“Neodol” 25, Shell Chemical Co.) | |
| Isopropyl palmitate | 5.9 |
| Polyethylene/acrylic acid copolymer | 2 |
| (AC-540 Polyethylene, Allied Chemical Co.) | |
| Paraffin wax (M.P. = 165. degree. F.) | 1 |
| (“Aristowax” 165, Witco Chemical Corp.) | |
| Hydrogenated coconut oil | 4 |
| (“Cobee” 92, M.P. 92. degree. F., PVO | |
| International, Inc.) | |
| Mineral oil/lanolin alcohol mixture | 1 |
| (“Amerchol” L-101, Amerchol, Unit of CPC | |
| International, Inc.) | |
| Octyl dimethyl PABA (“Escalol” | 2.7 |
| 507 VanDyk and Co., Inc.) | |
| Propyl paraben (“Lexgard” P, Inolex Corp.) | .15 |
| Phase B | |
| Deionized water | 63 |
| Xanthan gum (“Keltrol”, Kelco Div., | .5 |
| Merck & Co., Inc.) | |
| Magnesium sulfate (USP, Mallinckrodt) | .15 |
| Methyl paraben (“Lexgard” M, Inolex Corp.) | .3 |
| Phase C | |
| Fragrance | .3 |

The lotion is made by heating Phase A to 180° F. with slow agitation, and heating Phase B in a separate vessel to 180° F. with moderate agitation. Phase B is added to Phase A with rapid agitation, and the resulting creamy mixture is cooled with mixing to 100° F. The fragrance is then added. The composition is a smooth, white, creamy lotion which is barely pourable at room temperature. The lotion has a slightly oily feel.

The composition is tested for erythema protection on human volunteers using a 150 watt xenon arc solar simulator (available from Solar Light Company, Philadelphia, Pa. 19126). The composition (0.12 g) was applied to a 60 cm² area on the volar portion of the forearm, providing coverage of about 2.0 mg/cm². The amount of exposure necessary to elicit a minimal erythema response (minimum erythemal dose, MED) on the treated area was compared to the amount of exposure eliciting a MED on an untreated control area. This ratio is called the “protection factor.” The protection factor for this composition was four, (i.e., the average person can withstand four times the amount of erythema radiation with the composition than without it.)

The protection factor is changed by adjusting the level of ultraviolet light-absorber. The changes in the total weight can be compensated for by changing the level of isopropyl palmitate or combinations of the other oil phase ingredients. The protection factor data listed below shows the arithmetic means of protection factors determined on six human subjects for various levels of octyl dimethyl PABA. The standard deviation from the mean was approximately one for each of the data points.

| Wt. % octyl dimethyl PABA | Protection Factor |
|---|---|
| 2.0 | 3.71 |
| 2.5 | 4.25 |
| 2.7 | 4.55 |
| 3.0 | 4.83 |
| 3.5 | 7.58 |
| 3.7 | 8.25 |

Information from VanDyk & Co., Inc. on octyl dimethyl PABA indicates that a 1% solution by weight transmits about 13% of the incoming erythemal ultraviolet radiation. This would provide minimal protection, but could be beneficial to people who tan very well and rarely burn. The preferred range of octyl dimethyl PABA in the lotion is from 1 percent to 4 percent. This amount would provide protection factors from 2 to 8+. However, more or less could be used for those who desire extreme protection or for those who want very little protection.

3.2b Oil Formulation With Isooctyl Acrylate:Acrylic Acid Copolymer (Mole Ratio=90:10)

A three liter resin flask is charged with 1000 parts isopropyl palmitate (IPP), 958 parts isooctyl acrylate, 42 parts acrylic acid, and 7.14 g of 70 percent benzoyl peroxide at room temperature. The reactor is sealed, stirring initiated, and the system degassed by pulling a vacuum. The vacuum is broken with nitrogen and a nitrogen blanket is maintained over the system for the remainder of the polymerization. The reaction mixture is heated to 60° C. with heat lamps in 0.5 hr., and the temperature is maintained at 60° C. (with an ice bath and later with heat lamps) for six hours. The resulting polymer is cooled to room temperature. A diluted sample of the water-white polymer (2 parts polymer mixture to 7 parts IPP) gave a Brookfield viscosity (Spindle 3, 30 rpm) of 1250 cps.

The copolymer as diluted above was incorporated into an oil-based composition containing the following ingredients:

| | Parts by |
|---|---|
| Ingredient | Weight |
| CoQ10 | 1 |
| Copolymer solution | 9 |
| Carnation mineral oil | 20 |
| (Saybolt Viscosity at | |
| 100. degree. F. = 65/ | |
| 75, Witco Chemical Corp.) | |
| Kaydol mineral oil (Saybolt | 50 |
| Viscosity at 100. degree. F. = 345/ | |
| 355, Witco Chemical Corp.) | |
| Isopropyl palmitate | 8.55 |
| C₁₂-C.sub.15 alcohols | 5 |
| Coconut oil (M.P. 76. degree. F. | 4 |
| “Cobee” 76, PVO International, | |
| Inc.) | |
| Lanolin (Anhydrous USP | 1 |
| grade, Robinson, Wagner Co., | |
| Inc.) | |
| Octyl dimethyl PABA | 2 |
| Propyl paraben | .15 |
| Fragrance | .3 |

The oil is made by warming all of the ingredients, except the fragrance, to 140° F. with slow agitation. The fragrance is added after the oil has cooled to about 100° F.

The composition is a smooth, clear, pale gold oil which is very easy to spread on the skin. The protection factor for this composition (as determined in Example 1) is three.

Four oils identical to that above except with 2 percent, 2.5 percent, 3 percent, and 3.5 percent octyl dimethyl PABA were prepared. Each of these oils was tested on six human volunteers to determine its protection factor using the solar simulator. The average protection factor ranged from 2.97 for the oil with 2 percent ultraviolet light absorber to 4.21 for the oil with 3.5 percent ultraviolet light absorber. An oil with 4 percent ultraviolet light absorber should meet the needs of most oil users who want considerable protection. It is not economically feasible to make a high screen oil due to the large amount of expensive ultraviolet absorber which would be required. Many oil users desire very minimal protection and oil compositions containing low levels of ultraviolet light absorber are generally acceptable.

### Example 5

Lipstick Formulations

A cosmetic lipstick composition was made according to the following formula

| | w/w % |
|---|---|
| CoQ10 | 1.00 |
| Synthetic wax | 8.50 |
| Paraffin wax | 2.00 |
| Triisostearyl citrate | 36.40 |
| Dimethicone | 1.40 |
| Cholesterol/lanasterol esters | 1.00 |
| Red iron oxides | 3.54 |
| D&C Red #7 Calcium Lake | 0.78 |
| FD&C Yellow #5 Aluminum Lake | 1.55 |
| Black iron oxides | 1.76 |
| Titanium dioxide | 2.37 |
| Lanolin oil | 20.30 |
| Moisturizing complex* | 0.25 |
| Phytosterol/octyldodecyl/ | 0.05 |
| lauryl glutamate | |
| Cyclomethicone | 20.00 |
| Polymer solution*** | 0.10 |
| Bis-diglyceryl | 15.00 |
| polyacyladipate | |
| Methyl glucose | 27.00 |
| sesquistearate | |
| Hydrogenated | 23.70 |
| polyisobutene | |
| Sodium PCA | 2.00 |
| Gingko extract | 1.00 |
| Sodium lactate/sodium | 1.00 |
| PCA/urea/hydrolyzed | |
| collagen/sodium phosphate | |
| Methyl paraben | 0.30 |
| Propyl paraben | 0.10 |
| Isocetyl stearate | 11.90 |
| Sodium hylauronate/ | 9.00 |
| hydrolyzed | |
| glycosaminoglycans | |
| Magnesium ascorbyl | 0.50 |
| phosphate | |
| Pseudoceramide | 1.00 |
| (Questamide H) | |
| Acrylates copolymer | 7.50 |

***A solution containing about 90 parts by weight of a copolymer containing about equal parts by weight of isobutyl methacrylate and isobornyl methacrylate and about 10 parts by weight of isododecane.
*Moisurizing complex

The lipstick composition is made by combining the synthetic wax, paraffin wax, triisostearyl citrate, dimethicone, and cholesteryl lanasterol with heat to cause the waxes to melt. The pigments, lanolin oil, moisturizing complex and phytosterol mixture were then added with stirring to mix the ingredients. The cyclomethicone and Dupont polymer are added last. The mixture is poured into molds and allowed to cool to room temperature.

### Example 6

Petroleum Jelly and Mineral Oil Formulations

Into a jacketed kettle is charged 25% of the deionized water employed in the formulation. Thereafter, dimethyl distearyl ammonium chloride, petrolatum, cetyl alcohol, isopropyl palmitate, dimethicone (dimethyl polysiloxane fluid, supplied as Dow Corning-DC 225 fluid), methyl and propyl paraben preservatives and glycerin are added. The mixture is slowly heated to a temperature between about 180° F. and 190° F. while agitating the mix with sufficient shear and turnover to obtain a homogeneous mixture. Agitation is continued for about 30 minutes.

Thereafter, the remaining water, at an addition temperature from about 50° F. to 70° F., is added slowly under continuous and increasing agitation. After all the water is added, the mixture has a resulting temperature of from 90° F. to 110° F. The perfume is thereafter added under agitation until a homogeneous lotion is produced.

The resulting skin care product is applied to the skin as needed to alleviate the symptoms of dry skin. The skin care composition thus formed is comprised of the following ingredients:

| Ingredients | Weight Percent |
|---|---|
| CoQ10 | 1.0 |
| Dimethyl distearyl | 5.0 |
| ammonium chloride* | |
| Petrolatum, U.S.P. | 4.0 |
| Glycerin | 4.0 |
| Isopropyl palmitate | 3.0 |
| Cetyl alcohol | 2.0 |
| Dimethicone (10 cst) | 0.25 |
| Fragrance | 0.05 |
| Paraben preservatives | 0.14 |
| Deionized water | 81.56 |
| Total | 100.00 |

*Tradename - Quaternium5, supplied by Sherex Chemical as Arosurf TA100

### Example 7

Formulation for Lip Balm

A lip balm is prepared with the following composition (by weight):

| Active ingredients: | |
|---|---|
| CoQ10 | 1.000% |
| octyl methoxycinnamate | 7.500% |
| benzophenone | 2.500% |
| dimethicone | 2.000% |
| Other ingredients: | |
| dioctyl adipate/octyl | 15.244% |
| stearate/octyl palmitate | |
| caprylic/capric triglycerides | 13.596% |
| microcrystalline wax | 11.712% |
| liposome composition | 10.560% |
| petrolatum | 9.024% |
| ozokerite | 7.680% |
| purified water | 6.680% |
| flavor | 4.000% |
| jojoba esters | 2.400% |
| behenoyl stearate | 2.400% |
| sodium borate | 1.360% |
| panthenol | 1.000% |
| squalane | 1.000% |
| cholesteryl/behenyl/ | 0.480% |
| octadecyl lauroyl glutamate | |
| methyl paraben | 0.288% |
| propyl paraben | 0.288% |
| ethyl paraben | 0.096% |
| butyl paraben | 0.096% |
| sodium saccharin | 0.096% |
| TOTAL: | 100.000% |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

What is claimed is:

1. A composition for treatment of acne and enhanced dermatological health comprising:
(i) an anti-acne therapeutic; and
(ii) a compound according to the formula: 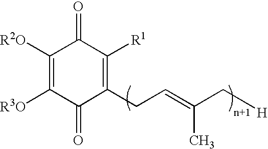
wherein
R¹, R² and R³ are members independently selected from substituted and unsubstituted C₁-C₆ alkyl groups; and
n is an integer from 0 to 19.

2. The composition of claim 1, wherein n is 9.

3. The composition of claim 2, wherein R¹, R² and R³ are methyl groups.

4. The composition of claim 3, wherein the compound is in a reduced form.

5. The composition of claim 1, wherein the anti-acne therapeutic is a member selected from a retinoid compound, azelaic acid, adapalene, salicylic acid, glycolic acid, benzoyl peroxide, erythromycin, and combinations thereof.

6. The composition of claim 1, wherein the anti-acne therapeutic is a member selected from a retinoid compound consisting of retinol, all trans-retinoic acid, 13-cis-retinoic acid, and combinations thereof.

7. The composition of claim 1, wherein the composition provides between about 1% to about 1000% better transdermal delivery of an anti-acne medication over formulations lacking a compound according to the formula: 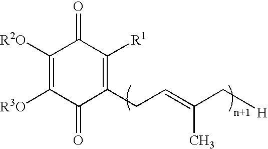
wherein
R¹, R² and R³ are members independently selected from substituted and unsubstituted C₁-C₆ alkyl groups; and
n is an integer from 0 to 19.

8. The composition of claim 7, wherein the composition provides between about 5% to about 500% better treatment of acne.

9. The composition of claim 7, wherein the composition provides between about 25% to about 100% better treatment of acne.

10. The composition of claim 1, further comprising a solubilizing agent of the formula: 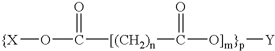
wherein:
X is a residue of a hydrophobic moiety, selected from the group consisting of sterols, tocopherols, and derivatives thereof;
Y is a residue of a hydrophilic moiety, selected from the group consisting of polyethers, polyalcohols, and derivatives thereof;
p is 1 or 2;
m is 0 or 1; and
n is an integer greater than or equal to 0; and
when p and m are equal to 1 and the hydrophobic moiety is cholesterol, n is greater than 4 and not equal to 8; and
when p and m are equal to 1, and the hydrophobic moiety is (+)-α-tocopherol, n is not equal to 2.

11. The composition of claim 10, wherein the hydrophobic moiety is a member selected from cholesterol, 7-dehydrocholesterol, campesterol, sitosterol, ergosterol, stigmasterol, α-tocopherol, β-tocopherol, γ-tocopherol, and δ-tocopherol, and the hydrophilic moiety is the polyether.

12. The composition of claim 11, wherein the polyether is a polyethylene glycol.

13. A composition providing enhanced performance of a topical antimicrobial ointment comprising:
(i) a therapeutically effective topical antimicrobial agent; and
(ii) a compound according to the formula: 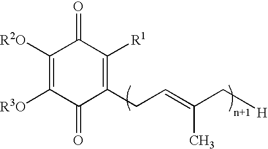
wherein
R¹, R² and R³ are members independently selected from substituted and unsubstituted C₁-C₆ alkyl groups; and
n is an integer from 0 to 19; and
wherein
the antimicrobial agent is other than a macrolide polyene antibiotic.

14. The composition of claim 13, wherein n is 9.

15. The composition of claim 14, wherein R¹, R² and R³ are methyl groups.

16. The composition of claim 15, wherein the compound is in a reduced form.

17. The composition of claim 13, wherein the antimicrobial agent is a member selected from alcohol, neosporin, polysporin, benzalkonium chloride, chlorhexidine, hexachlorophine, fusidic acid, neomycin, oxytetracyclin, mupirocin, flucloxacillin, acyclovir and combinations thereof.

18. The composition of claim 13, wherein the composition further comprises an analgesic.

19. The composition of claim 13, wherein the composition further comprises an anti-inflammatory agent.

20. The composition of claim 19, wherein the anti-inflammatory agent further comprises cortisone.

21. The composition of claim 13, further comprising a solubilizing agent of the formula: 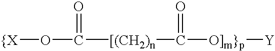
wherein:
X is a residue of a hydrophobic moiety, selected from the group consisting of sterols, tocopherols, and derivatives thereof;
Y is a residue of a hydrophilic moiety, selected from the group consisting of polyethers, polyalcohols, and derivatives thereof;
p is 1 or 2;
m is 0 or 1; and
n is an integer greater than or equal to 0; and
when p and m are equal to 1 and the hydrophobic moiety is cholesterol, n is greater than 4 and not equal to 8; and
when p and m are equal to 1, and the hydrophobic moiety is (+)-α-tocopherol, n is not equal to 2.

22. The composition of claim 21, wherein the hydrophobic moiety is a member selected from cholesterol, 7-dehydrocholesterol, campesterol, sitosterol, ergosterol, stigmasterol, α-tocopherol, β-tocopherol, γ-tocopherol, and δ-tocopherol, and the hydrophilic moiety is the polyether.

23. The composition of claim 22, wherein the polyether is a polyethylene glycol.

24. The composition of claim 13, wherein the performance of the antimicrobial agent is enhanced by between about 1% to about 1000% over formulations lacking a compound according to the formula: 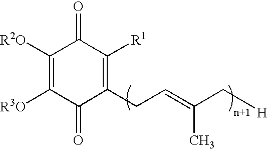
wherein
R¹, R² and R³ are members independently selected from substituted and unsubstituted C₁-C₆ alkyl groups; and
n is an integer from 0 to 19.

25. The composition of claim 24, wherein the performance of the antimicrobial agent is enhanced by between about 5% to about 500%.

26. The composition of claim 24, wherein the performance of the antimicrobial is enhanced by between about 25% to about 100%.

27. A sunscreen composition providing enhanced performance of a sunscreening agent comprising:
(i) an effective sunscreening agent; and
(ii) a compound according to the formula: 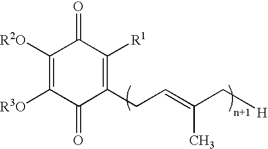
wherein
R¹, R² and R³ are members independently selected from substituted and unsubstituted C₁-C₆ alkyl groups; and
n is an integer from 0 to 19.

28. The sunscreen composition of claim 27, wherein n is 9.

29. The sunscreen composition of claim 28, wherein R¹, R¹ and R³ are methyl groups.

30. The sunscreen composition of claim 29, wherein the compound is in a reduced form.

31. The sunscreen composition of claim 27, further comprising a solubilizing agent of the formula: 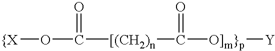
wherein:
X is a residue of a hydrophobic moiety, selected from the group consisting of sterols, tocopherols, and derivatives thereof;
Y is a residue of a hydrophilic moiety, selected from the group consisting of polyethers, polyalcohols, and derivatives thereof;
p is 1 or 2;
m is 0 or 1; and
n is an integer greater than or equal to 0; and
when p and m are equal to 1 and the hydrophobic moiety is cholesterol, n is greater than 4 and not equal to 8; and
when p and m are equal to 1, and the hydrophobic moiety is (+)-α-tocopherol, n is not equal to 2.

32. The sunscreen composition of claim 31, wherein the hydrophobic moiety is a member selected from cholesterol, 7-dehydrocholesterol, campesterol, sitosterol, ergosterol, stigmasterol, α-tocopherol, β-tocopherol, γ-tocopherol, and δ-tocopherol, and the hydrophilic moiety is the polyether.

33. The sunscreen composition of claim 32, wherein the polyether is a polyethylene glycol.

34. The sunscreen composition of claim 27, wherein the sunscreen composition is comprised within a formulation for a product that is a member selected from a cosmetic foundation, a lip balm, and a lipstick.

35. A composition for facial cleansing comprising:
(i) an effective facial cleanser; and
(ii) a compound according to the formula: 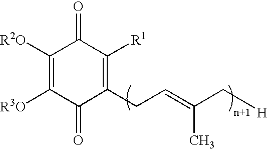
wherein
R¹, R² and R³ are members independently selected from substituted and unsubstituted C₁-C₆ alkyl groups; and
n is an integer from 0 to 19; and
wherein
the composition further comprises a solubilizing agent of the formula: 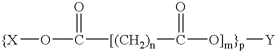
wherein:
X is a residue of a hydrophobic moiety, selected from the group consisting of sterols, tocopherols, and derivatives thereof;
Y is a residue of a hydrophilic moiety, selected from the group consisting of polyethers, polyalcohols, and derivatives thereof;
p is 1 or 2;
m is 0 or 1; and
n is an integer greater than or equal to 0; and
when p and m are equal to 1 and the hydrophobic moiety is cholesterol, n is greater than 4 and not equal to 8; and
when p and m are equal to 1, and the hydrophobic moiety is (+)-α-tocopherol, n is not equal to 2.

36. The composition of claim 35, wherein n is 9.

37. The composition of claim 36, wherein R¹, R² and R³ are methyl groups.

38. The composition of claim 37, wherein the compound is in a reduced form.

39. The composition of claim 35, wherein the hydrophobic moiety is a member selected from cholesterol, 7-dehydrocholesterol, campesterol, sitosterol, ergosterol, stigmasterol, α-tocopherol, β-tocopherol, γ-tocopherol, and δ-tocopherol, and the hydrophilic moiety is the polyether.

40. The composition of claim 39, wherein the polyether is a polyethylene glycol.

41. The composition of claim 35, wherein the composition is comprised within a formulation for a product selected from the group consisting of a facial mask and a bar soap.

42. The composition of claim 41, wherein the facial mask further comprises a glacial silt, or a glacial clay.

43. The composition of claim 42, wherein the glacial silt is comprised of silt particles whose average diameter is in a range from between about 0.004 mm. to about 0.063 mm.

44. A method for delivering a composition comprising a compound according to the formula: 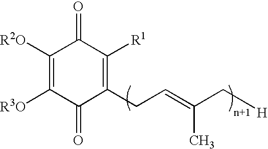
wherein
R¹, R² and R³ are members independently selected from substituted and unsubstituted C₁-C₆ alkyl groups;
and n is an integer from 0 to 19; and
comprising:
(i) providing the compound via a transdermal delivery system.

45. The method of claim 44, wherein n is 9.

46. The method of claim 45, wherein R¹, R² and R³ are methyl groups.

47. The method of claim 46, wherein the compound is in a reduced form.

48. The method of claim 44, wherein the transdermal delivery system is a dermal patch.

49. The method of claim 44, wherein the composition further comprises a solubilizing agent of the formula: 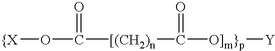
wherein:
X is a residue of a hydrophobic moiety, selected from the group consisting of sterols, tocopherols, and derivatives thereof;
Y is a residue of a hydrophilic moiety, selected from the group consisting of polyethers, polyalcohols, and derivatives thereof;
p is 1 or 2;
m is 0 or 1; and
n is an integer greater than or equal to 0; and
when p and m are equal to 1 and the hydrophobic moiety is cholesterol, n is greater than 4 and not equal to 8; and
when p and m are equal to 1, and the hydrophobic moiety is (+)-α-tocopherol, n is not equal to 2.

50. The method of claim 49, wherein the hydrophobic moiety is a member selected from cholesterol, 7-dehydrocholesterol, campesterol, sitosterol, ergosterol, stigmasterol, α-tocopherol, β-tocopherol, γ-tocopherol, and δ-tocopherol, and wherein the hydrophilic moiety is the polyether.

51. The method of claim 50, wherein the polyether is a polyethylene glycol.

52. A composition for reducing skin inflammation and firming skin tone, comprising:
(i) a compound according to the formula: 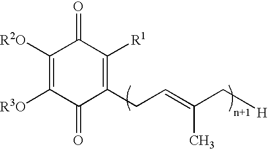
wherein
R¹, R² and R³ are members independently selected from substituted and unsubstituted C₁-C₆ alkyl groups; and
n is an integer from 0 to 19.

53. The composition of claim 52, wherein n is 9.

54. The composition of claim 53, wherein R¹, R² and R³ are methyl groups.

55. The composition of claim 54, wherein the compound is in a reduced form.

56. The composition of claim 52, wherein the composition further comprises cortisone.

57. The composition of claim 52, wherein the skin inflammation is associated with an inflammatory dermatosis.

58. The composition of claim 57, wherein the inflammarory dermatosis is Acne Rosacea.

59. The composition of claim 52, wherein the composition further comprises a solubilizing agent of the formula: 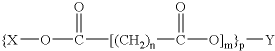
wherein:
X is a residue of a hydrophobic moiety, selected from the group consisting of sterols, tocopherols, and derivatives thereof;
Y is a residue of a hydrophilic moiety, selected from the group consisting of polyethers, polyalcohols, and derivatives thereof;
p is 1 or 2;
m is 0 or 1; and
n is an integer greater than or equal to 0; and
when p and m are equal to 1 and the hydrophobic moiety is cholesterol, n is greater than 4 and not equal to 8; and
when p and m are equal to 1, and the hydrophobic moiety is (+)-α-tocopherol, n is not equal to 2.

60. The composition of claim 59, wherein the hydrophobic moiety is a member selected from cholesterol, 7-dehydrocholesterol, campesterol, sitosterol, ergosterol, stigmasterol, α-tocopherol, β-tocopherol, γ-tocopherol, and δ-tocopherol and the hydrophilic moiety is the polyether.

61. The composition of claim 60, wherein the polyether is a polyethylene glycol.

62. A cosmetic foundation comprising:
(i) a compound according to the formula: 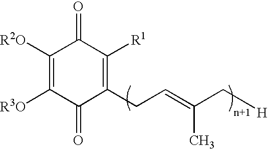
wherein
R¹, R² and R³ are members independently selected from substituted and unsubstituted C₁-C₆ alkyl groups; and
n is an integer from 0 to 19.

63. The cosmetic foundation of claim 62, wherein n is 9.

64. The cosmetic foundation of claim 63, wherein R¹, R² and R³ are methyl groups.

65. The cosmetic foundation of claim 64, wherein the compound is in a reduced form.

66. The cosmetic foundation of claim 62, wherein the composition further comprises a solubilizing agent of the formula: 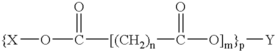
wherein:
X is a residue of a hydrophobic moiety, selected from the group consisting of sterols, tocopherols, and derivatives thereof;
Y is a residue of a hydrophilic moiety, selected from the group consisting of polyethers, polyalcohols, and derivatives thereof;
p is 1 or 2;
m is 0 or 1; and
n is an integer greater than or equal to 0; and
when p and m are equal to 1 and the hydrophobic moiety is cholesterol, n is greater than 4 and not equal to 8; and
when p and m are equal to 1, and the hydrophobic moiety is (+)-α-tocopherol, n is not equal to 2.

67. The cosmetic foundation of claim 66, wherein the hydrophobic moiety is a member selected from cholesterol, 7-dehydrocholesterol, campesterol, sitosterol, ergosterol, stigmasterol, α-tocopherol, β-tocopherol, γ-tocopherol, and δ-tocopherol, and the hydrophilic moiety is the polyether.

68. The cosmetic foundation of claim 67, wherein the polyether is a polyethylene glycol.

69. A formulation comprising:
(i) a compound according to the formula: 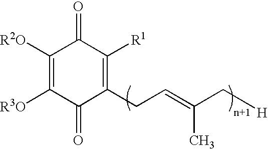
wherein
R¹, R¹ and R³ are members independently selected from substituted and unsubstituted C₁-C₆ alkyl groups; and
n is an integer from 0 to 19; and
(ii) a hyaluronic acid.

70. The formulation of claim 69, wherein n is 9.

71. The formulation of claim 70, wherein R¹, R² and R³ are methyl groups.

72. The formulation of claim 71, wherein the compound is in a reduced form.

73. The formulation of claim 69, wherein the compound according to the formula: 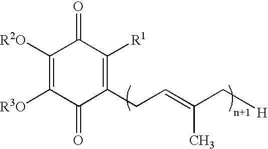
wherein
R¹, R² and R³ are members independently selected from substituted and unsubstituted C₁-C₆ alkyl groups; and
n is an integer from 0 to 19; and
improves penetration of the hyaluronic acid into skin cells.

74. The formulation of claim 69, wherein the hyaluronic acid penetrates skin cells between about 1% to 1000% better than hyaluronic acid comprising a lotion lacking a compound according to the formula: 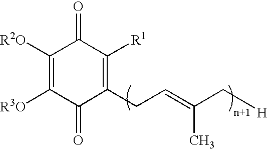
wherein
R¹, R² and R³ are independently selected from substituted and unsubstituted C₁-C₆ alkyl groups; and
n is an integer from 0 to 19.

75. A composition for skin protection comprising:
(i) a compound according to the formula: 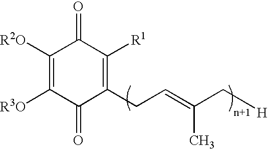
wherein
R¹, R² and R³ are members independently selected from substituted and unsubstituted C₁-C₆ alkyl groups; and
n is an integer from 0 to 19; and
(ii) a compound that is a member selected from mineral oil and petroleum jelly.

76. The composition of claim 75, wherein n is 9.

77. The composition of claim 76, wherein R¹, R² and R³ are methyl groups.

78. The composition of claim 77, wherein the compound is in a reduced form.

79. A composition for protecting and enhancing the health of the skin of the lips comprising:
(i) a compound according to the formula: 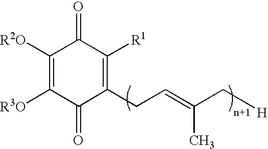
wherein
R¹, R² and R³ are members independently selected from substituted and unsubstituted C₁-C₆ alkyl groups; and
n is an integer from 0 to 19.

80. The composition of claim 79, wherein n is 9.

81. The composition of claim 80, wherein R¹, R² and R³ are methyl groups.

82. The composition of claim 81, wherein the compound is in a reduced form.

83. The composition of claim 79, wherein the composition is in a form that is a member selected from a lipstick and a lip balm.

84. A composition for skin cream comprising:
(i) a compound according to the formula: 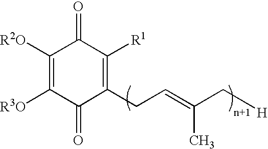
wherein
R¹, R² and R³ are members independently selected from substituted and unsubstituted C₁-C₆ alkyl groups; and
n is an integer from 0 to 19; and
(ii) vitamin C.

85. The composition of claim 84, wherein n is 9.

86. The composition of claim 85, wherein R¹, R² and R³ are methyl groups.

87. The composition of claim 86, wherein the compound is in a reduced form.

88. The composition of claim 84, wherein the formula further comprises at least one antioxidant selected from the group consisting of vitamin A, vitamin D, vitamin E, astaxanthin, alpha-lipoic acid, zeaxanthin, lutein, flavonoid and pycnogenol.
